(19) United States
(12) Patent Application Publication
(10) Pub. No.: US 2014/0066811 A1
(43) Pub. Date: Mar. 6, 2014

(54) POSTURE MONITOR

(71) Applicant: Ben Garney, Eugene, OR (US)

(72) Inventor: Ben Garney, Eugene, OR (US)

(21) Appl. No.: 14/019,180

(22) Filed: Sep. 5, 2013

Related U.S. Application Data
(60) Provisional application No. 61/697,220, filed on Sep. 5, 2012.

(51) Int. Cl. A61B 5/107

(52) U.S. Cl. CPC A61B 5/1079
USPC 600/587

(57) ABSTRACT

Disclosed is a method for detecting the body posture using sensors and supporting software, analyzing detected posture for deficiencies versus customizable parameters, recording posture trends over time, and training a user to adopt correct posture through dynamic feedback methods.

### CROSS REFERENCE TO RELATED APPLICATION

This application claims the benefit of U.S. Provisional Application No. 61/697,220, filed Sep. 5, 2012, which is herein incorporated by reference in its entirety.

### FIELD

This application concerns methods and devices for modifying behavior to improve posture.

### BACKGROUND

A person's body posture may be improved through monitoring and feedback, leading to behavior modification in the form of better posture. Monitoring is typically accomplished by locating one or more signaling devices on or resting against the subject so that they can detect and react to motion of the subject. Typical devices include pressure sensors, accelerometers, and reflectors. There are disadvantages in terms of cost and time and complexity to set up in having to locate signaling devices next to the subject. Feedback can take various forms but can suffer from being too late, susceptible to error, and/or ineffective relative to the user.

### SUMMARY

In certain embodiments of the present disclosure, a method for improving posture is implemented. That method includes the steps of acquiring data about a subject using sensing equipment, making a comparison to threshold posture data, and reporting results of the comparison. The sensing equipment can be some distance from the subject. That distance can be at least two feet away from the subject.

In another embodiment, reporting can include an audio signal.

In another embodiment, reporting can include computer graphics on an electronic display.

In another embodiment, reporting can include at least a portion of an image of the subject.

In another embodiment, at least a portion of the results is updated at some frequency. That frequency can be at least once per second.

In another embodiment, results can be reported to a user other than the subject.

In another embodiment, a posture program can execute one or more steps in a method to improve posture. That program can run in a multi-threaded computational environment, and that program can run in background while another program runs in foreground.

In another embodiment, the data acquired can be image data from which is extracted scalar posture data.

In another embodiment, a method for improving posture can ensure that two or more body locations all must be positioned correctly to avoid reporting a posture deficiency.

In another embodiment, at least a part of the hardware or software of a posture estimation or feature extraction system can contribute to any of acquiring raw data, processing that raw data, and/or extracting posture values from the data.

### BRIEF DESCRIPTION OF THE DRAWINGS

FIG. 1 is a schematic diagram showing a side elevation view of an exemplary environment for the method and system described herein.

FIG. 2 is a schematic diagram showing a top plan view of the same exemplary environment as in FIG. 1.

FIG. 3 is a conceptual diagram illustrating a feedback loop in an example of the method and system described herein.

FIG. 4 is a block diagram of an example of the method and system described herein.

FIG. 5 is a flowchart of an example of the method and system described herein.

FIG. 6 is a block diagram of a computer vision example of the method and system described herein.

FIG. 7 is a flowchart of a computer vision example of the method and system described herein.

FIG. 8 is a block diagram of a computer vision example of the method and system described herein that includes color and depth sensing.

FIG. 9 is a flowchart of a computer vision example of the method and system described herein that includes color and depth sensing.

FIG. 10 is a schematic diagram of example scalar values used in an example of the method and system described herein.

FIG. 11 is a schematic diagram of an exemplary user interface for providing feedback to a subject using a schematic representation of the subject.

FIG. 12 is a schematic diagram of an exemplary user interface for providing feedback to a subject using an image of the subject.

FIG. 13 is a block diagram of an exemplary computing environment suitable for implementing the technologies described herein.

### DETAILED DESCRIPTION

As used herein, the singular forms “a,” “an,” and “the” refer to one or more than one, unless the context clearly dictates otherwise.

As used herein, the term “includes” means “comprises.” For example, a device that includes or comprises A and B contains A and B but may optionally contain C or other components other than A and B. A device that includes or comprises A or B may contain A or B or A and B, and optionally one or more other components such as C.

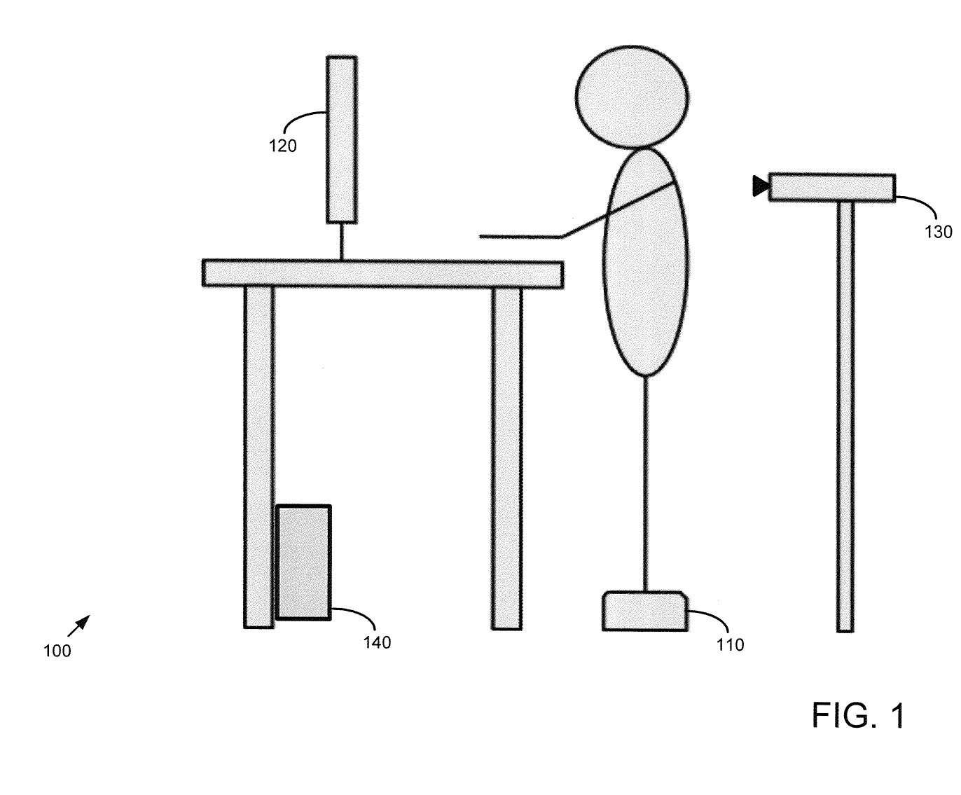

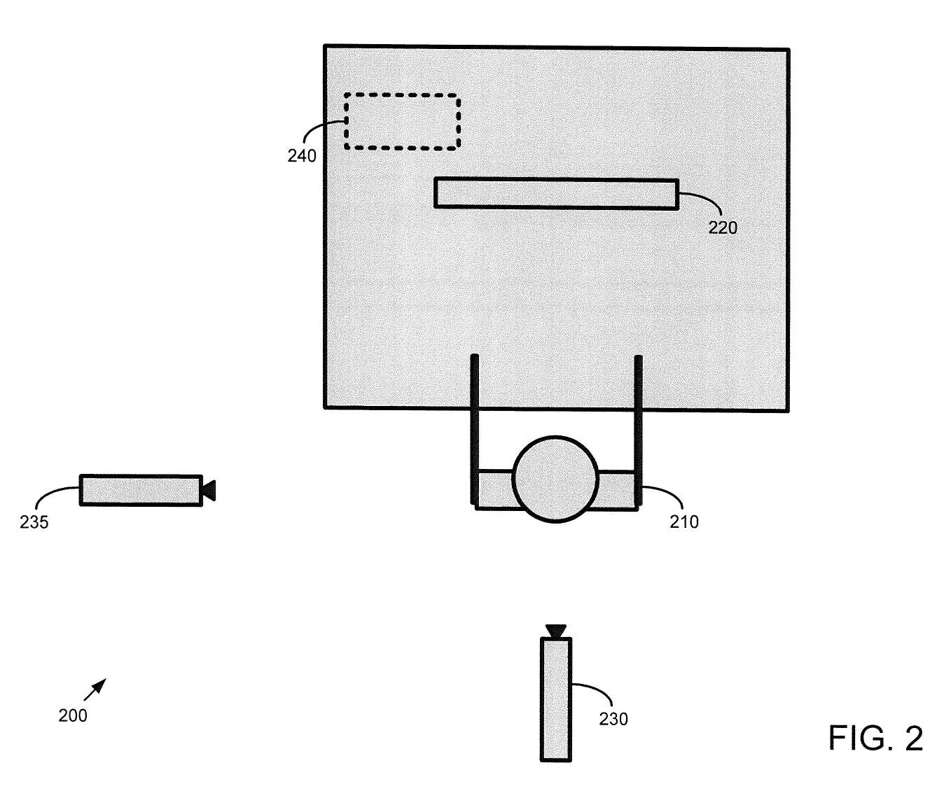

Referring first to FIGS. 1 and 2, there is shown an exemplary environment for the disclosure herein. FIG. 1 shows a side elevation view of the environment 100 with a subject 110 positioned facing a computer monitor 120. The subject is shown standing, but the subject might also be in other positions, including but not limited to sitting or kneeling. A computer monitor is shown, but in other embodiments the subject might be facing other work or leisure devices, including but not limited to a painting easel, a typewriter, or a stovetop. In another embodiment, the subject might not be facing any particular device or work surface. A sensor 130 is positioned so as to be capable of sensing the subject. The sensor 130 shown is a camera positioned longitudinally rearward of and laterally aligned with the subject. Other sensors capable of acting at a distance and other positions are equally possible, including but not limited to a color camera, a gray scale camera, stereo cameras, a range finder, and stereo range finders. Other possible sensor positions include but are not limited to above, in front of the subject (in the longitudinal direction), to the side of the subject (in the lateral direction), and below the subject. Not shown in FIG. 1 is a second camera. In an embodiment, a sensor system is capable of detecting a subject at a distance. That distance can be in a range from 1 inch to 100 yards. Preferred distances are between 2 feet and 20 feet, and most preferred distances are 3 feet, 6 feet or 10 feet.

Also, in another example, multiple sensors can be located at different positions, yielding multiple simultaneous overlapping or non-overlapping fields of view or viewpoints to one or more subjects. Data from multiple cameras and/or detectors can be combined before, during, and/or after other processing steps disclosed herein. Overlapping image data can be aligned and/or fused. In an example, there can be multiple subjects monitored individually by the same method or system. Subjects can be confined to relatively small volumes spanning less than 3 feet, or subjects can be monitored while changing position and moving throughout larger volumes, including but not limited to volumes spanning 10 yards or 100 yards or more.

A computing device 140, such as server computers, desktop computers, laptop computers, notebook computers, handheld devices, netbooks, tablet devices, mobile devices, PDAs, and other types of computing devices, can be located near the subject as part of a posture monitoring system or used to implement a posture monitoring method, but other computing resources can equally be used, including but not limited to a portable computing device carried or worn by the subject or a remote computing device accessed via a computer network connection.

FIG. 2 shows a top plan view of the same embodiment as FIG. 1. The subject 210 is again facing a computer monitor 220 with a local computing device 240 out of sight. In FIG. 2, two cameras are shown. Camera 230 corresponds to camera 130 of FIG. 1. Camera 235 is also shown in FIG. 2 and is laterally offset to the left of and longitudinally aligned with the subject. The cameras 230, 235 are positioned so as to view the subject from behind and from the side.

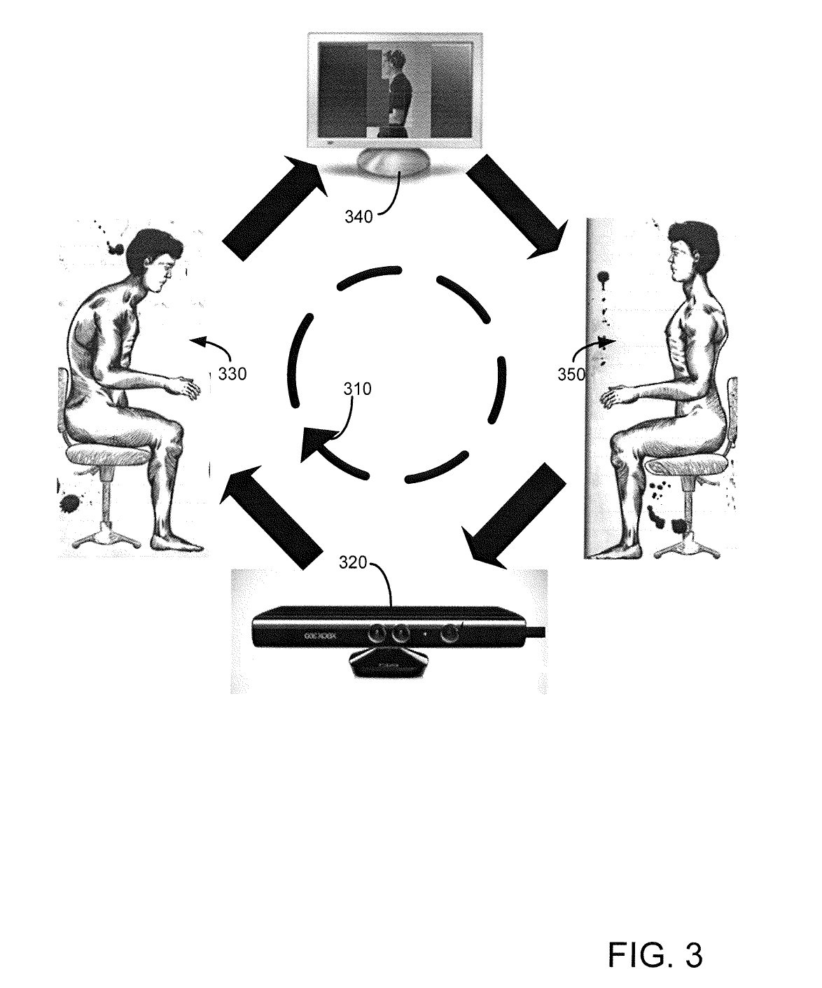

Referring to FIG. 3, the approaches described herein provide for a repeating activity that creates a feedback loop 310. The feedback loop can include a sensor 320 sensing the posture of a subject 330 and guiding that subject with a visual stimulus 340 to achieve improved posture 350 and then repeating the loop again beginning with the sensor 320. The subject is shown in this example perceiving the stimulus visually, but other equally possible forms of stimulus include an audio stimulus or a tactile stimulus as an alternative to or in addition to a visual stimulus.

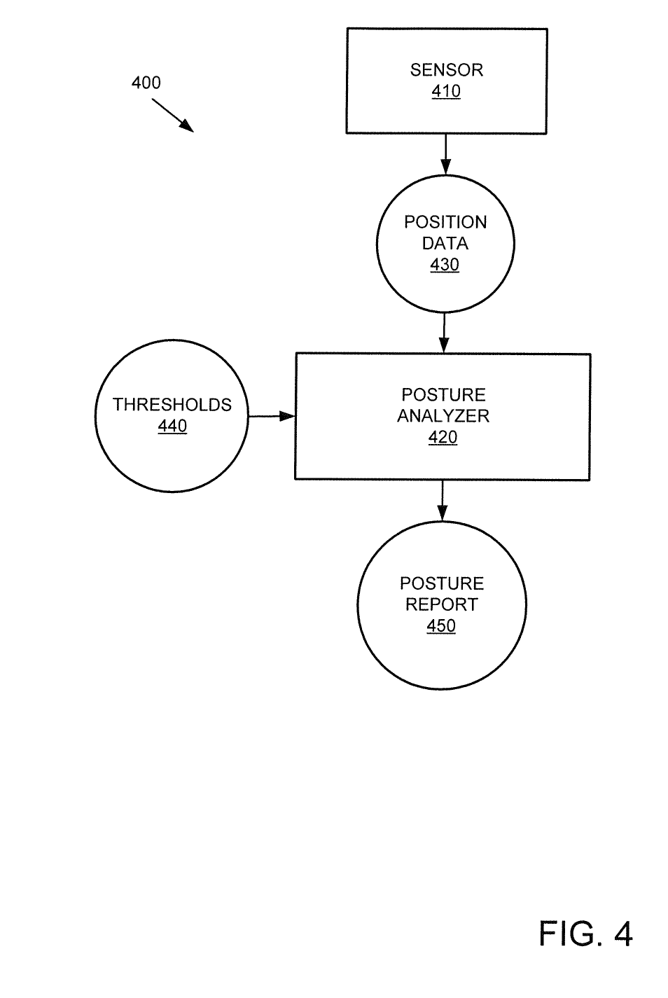

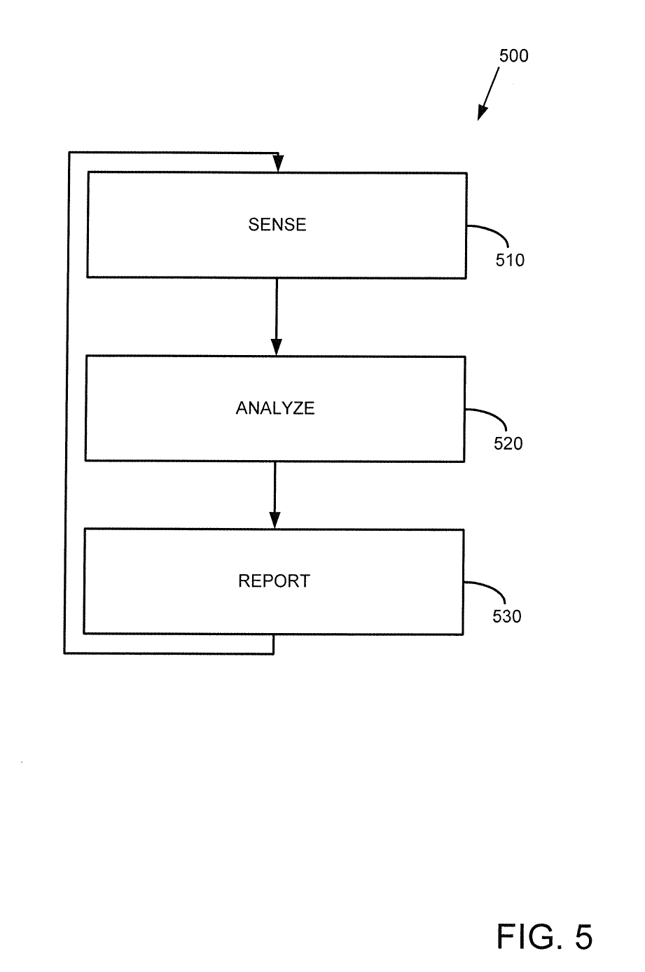

Referring to the embodiment shown in FIGS. 4 and 5, the system 400 includes a sensor 410 and a posture analyzer 420. The sensor can include sensing equipment generally and can be a one-dimensional range detector or a more complex image forming camera. Examples of detector types include but are not limited to detection of electromagnetic (for example visible light, infrared light, and radar) and acoustic energy. Further examples of detector types include but are not limited to: LIDAR detectors, millimeter wave radar detectors, and real time magnetic resonance imagers. A sensor may be passive with just an energy detector, or a sensor may be active and include one or more energy emitters. A sensor may be a single sensor or an array of two or more component sensors. A sensor may include computing resources for on-board processing of the raw sensors data. In the embodiment shown, the posture analyzer takes as input position data 430 from the sensor. Examples of position data include but are not limited to raw or processed scalar position values and raw or processed images. Light images can be two- or three-dimensional, color or gray scale, focused or plenoptic. The posture analyzer provides computing resources. It may be dedicated hardware and software or it may be a program running on a computing platform. A program may run alone on hardware or a program may time share or multi-process with other programs in a multi-threaded computational environment, in which case some programs may run in background while other programs run in background.

The posture analyzer takes as additional input thresholds 440. Thresholds include threshold posture data. In an embodiment, thresholds are scalar values that allow the analyzer to assess the position data, determine if the detected posture meets a predetermined acceptable posture, i.e. a healthy posture, and generate a posture report 450. In an embodiment, a posture report can include posture deficiencies, which are determinations of unhealthy posture. In an embodiment, posture deficiencies can be reported as part of the posture report and/or as any type of stimuli to change posture. The posture analyzer may process the position data and/or the thresholds before comparing the two types of data. Processing may include analyzing the received position data to extract values for the positions of body parts of the subject, including but not limited to displacement and rotation of the head, shoulders, arms, back, chest, pelvis, legs, feet, upper body, and lower body. In an embodiment, at least two body parts both must be positioned correctly to avoid reporting a posture deficiency. Basing results on the position of more than one body part can reduce errors in assessing posture. When posture related to multiple body parts or to the whole body posture is tracked, a subject is less likely to fall into incorrect postures that satisfy a single threshold value.

In an embodiment, threshold data is customized by the subject or by another user so that the threshold data can account for the body type and/or physical limitations of different subjects. If a subject has physical limitations, rather than simply bad habits or a lack of conditioning or strength, then user-selectable constraints can be relaxed or removed.

In an embodiment, a subject's posture is tracked and the thresholds for feedback are configurable. In this embodiment, a physical therapist and/or algorithm can set achievable goals for the subject, leading to gradual improvement, rather than requiring 100% compliance from the start.

Thresholds provide ranges to which to compare the extracted position data and determine and report better or worse posture over time. As an example, a posture report can be a binary positive or negative signal delivered through visual, oral, or tactile means. In an embodiment, a remote controlled haptic feedback device on the subject's body can be used. As another example, a posture report can be input to a user interface that communicates behavioral stimuli in the form of indications of correct posture or of posture deficiencies. As another example, a posture report can be a set of data in a file, including but not limited to tables of deficiencies or lack thereof over time, without or without additional automated explanatory notes. A posture report may be delivered in real time and/or it may be stored for later evaluation. A posture report may be intended for delivery to the subject and/or to another user, including but not limited to a therapist or a health/safety evaluator.

In the embodiment of FIGS. 4 and 5, the flowchart of FIG. 5 shows a method suitable to the system of FIG. 4. In the step “sense” 510, sensing can be implemented by the sensor 410 and delivers as output position data 430 to the posture analyzer 420. In an embodiment, sensing includes acquiring data about a subject using sensing equipment. In the step “analyze” 520, analyzing can be implemented by the sensor 410 and by the posture analyzer 420. Analyzing takes as input position data 430 and thresholds 440, performs analysis of these data, and delivers input to a posture report 450. In step “report” 530, reporting also can be implemented by the posture analyzer 420 and delivers as output the posture report 450.

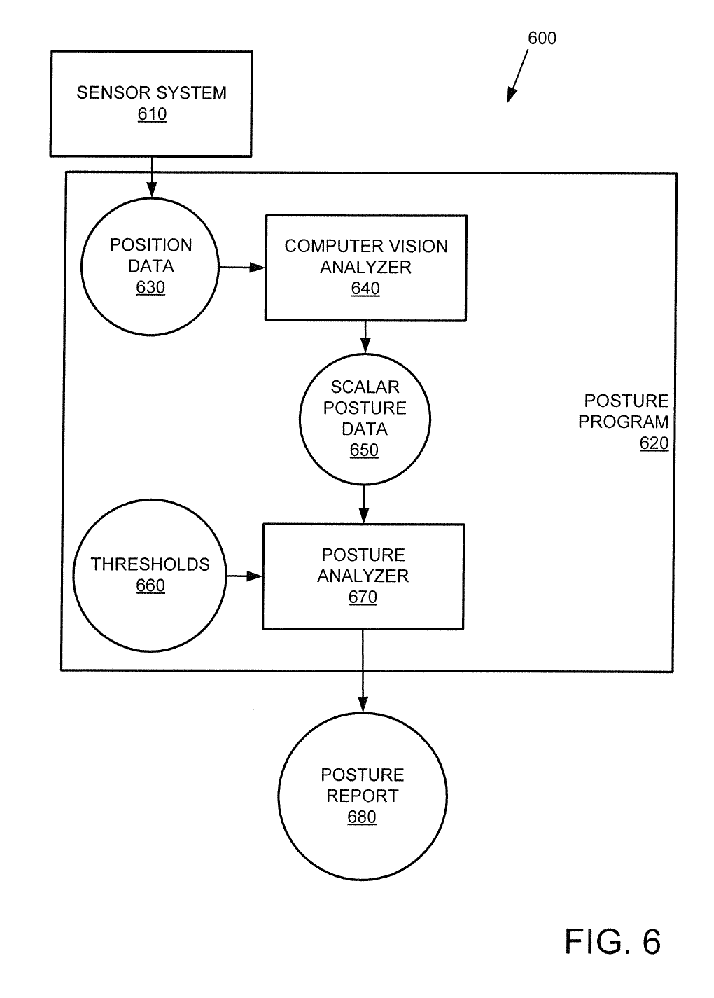

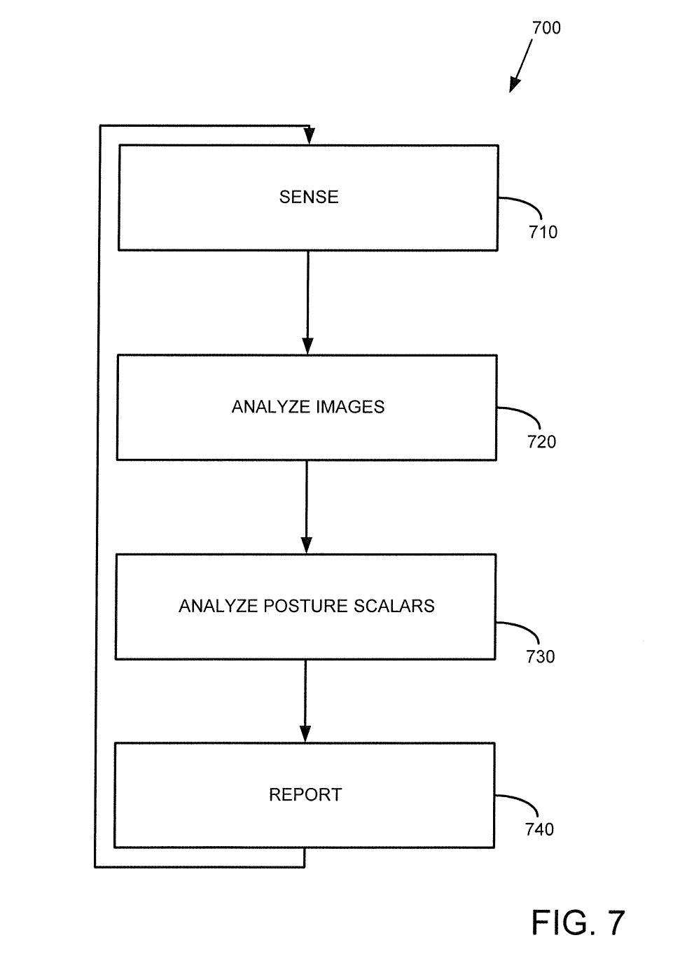

Referring to the embodiment shown in FIGS. 6 and 7, the system 600 includes separate computer vision 640 and posture 670 analyzers. These analyzers can be distinct programming objects within a posture program 620. The computer vision analyzer can receive position data in the form of raw or processed images and then process those images to segment them or to extract features and then compute position values needed for posture analysis. One level of analysis can be feature extraction and can include locating within an image one or more portions of each subject's body and then assigning selected and/or computed image data separately to the identified bodies or body portions. A second level of analysis can be calculation of scalar posture data 650 taking as input the segmented feature data from the first level of analysis. Scalar posture data refers to individual scalar values or measurements relevant to posture assessment.

In the embodiment of FIGS. 6 and 7, the flowchart of FIG. 7 shows a method suitable to system of the block diagram of FIG. 6. In the step “sense” 710, sensing can be implemented by the sensor system, 610 and delivers as output position data 630 to the computer vision analyzer 640. In the step “analyze images” 720, analyzing images can be implemented by the sensor system 610 and by the computer vision analyzer 640. Analyzing images takes as input position data 630 which can include but is not limited to image data, performs analysis of these data, and delivers scalar posture data 650 as input to the posture analyzer 670. In an embodiment, analyze images 720 can include full-field image processing, such as but limited to dark field noise correction, background subtraction, masking, and brightness and contrast adjustment. Analyze images 720 can also include segmented image operations and processes, for example computer vision, blob detection, Haar pyramids analysis, feature extraction, or pose estimation, to locate objects within an image such as subject bodies and body parts. In an embodiment, a goal of analyze images 720 is to reduce sets of position data including image data, which can be multidimensional in three spatial dimensions and in time, to scalar expressions of subject body part locations, herein referred to as scalar posture data 650. In the step “analyze posture scalars” 730, analyzing posture scalars can be implemented by the posture analyzer 670. Analyzing posture scalars takes as input scalar position data 650 and thresholds 660, performs analysis of these data, and delivers input to a posture report 680. In an embodiment, analysis of scalar posture data includes comparison with scalar threshold values or thresholds to arrive at conclusions as to whether measured posture parameters are acceptable or not. In step “report” 740, reporting also can be implemented by the posture analyzer 670 and delivers as output the posture report 680. At least some of the conclusions of an analysis of posture scalars can be included in a posture report.

In an embodiment, at least a part of the hardware or software of a Microsoft® Kinect® system provides at least part of the sensor 610 and/or the computer vision analyzer 640. In an embodiment, a Microsoft® Kinect® system performs one or more of the following functions: sensing 710, acquiring images of the subject, performing image segmentation, calculating position data 630. In another embodiment, related alternatives to Microsoft® Kinect® can be used, including but not limited to other computer vision, feature extraction, or posture estimation techniques and/or software. In still other embodiments, other methods of image segmentation or feature extraction can be used, including but not limited to blob detection or Haar pyramids.

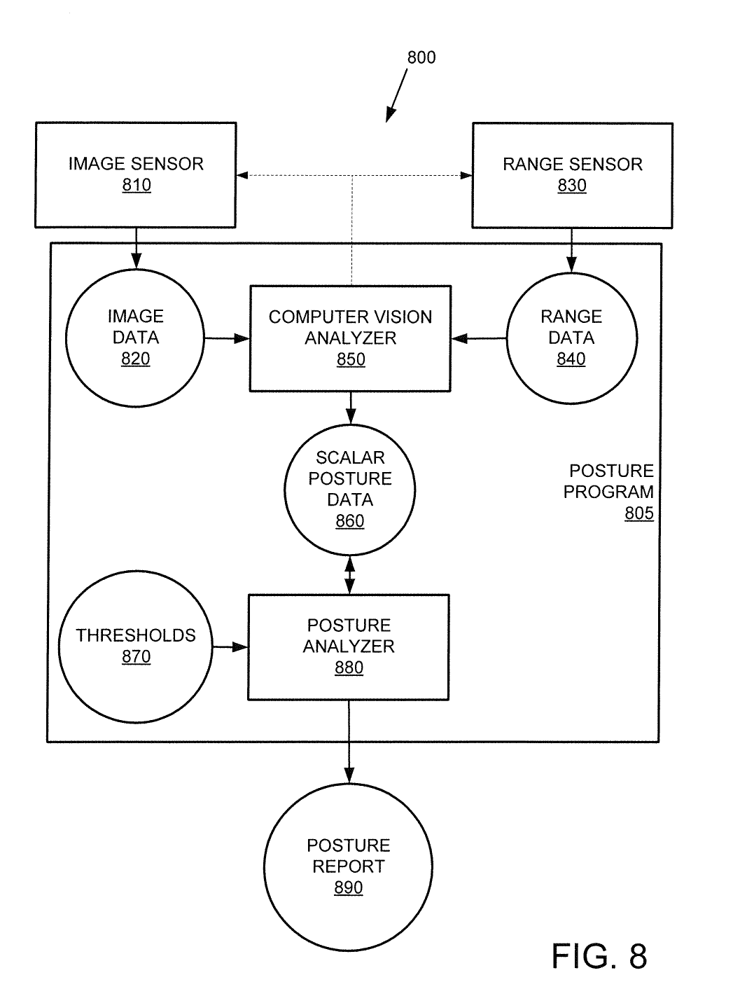

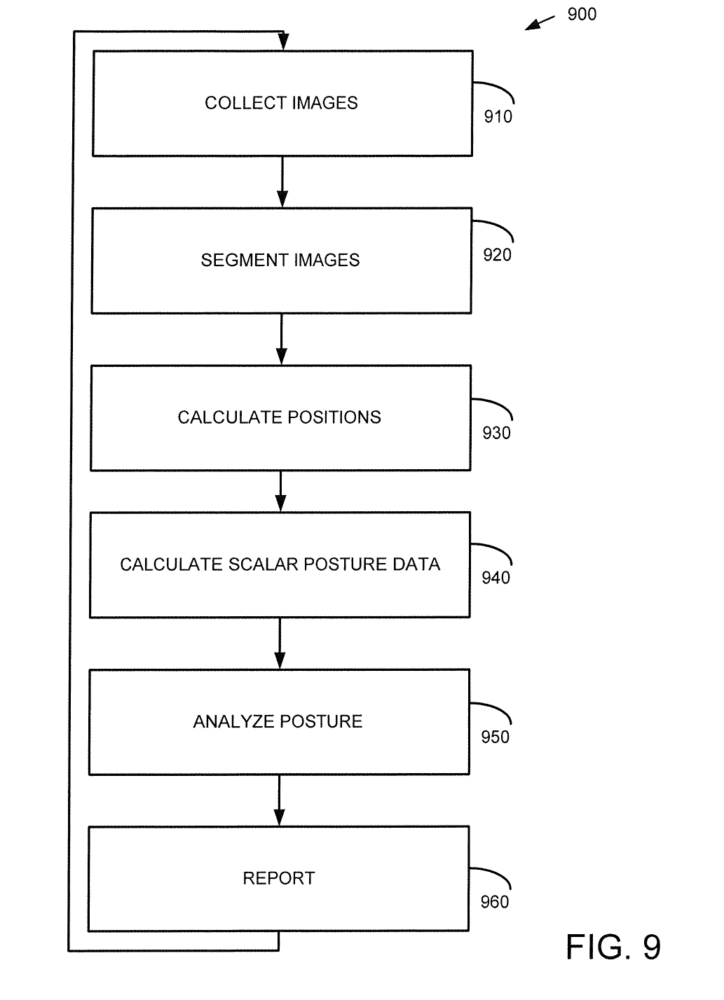

Referring to the embodiment shown in FIGS. 8 and 9, the system 800 includes separate image 810 and range 830 sensors that send input to a posture program 805. The image sensor 810 provides image data 820 used for segmentation, feature extraction, and/or locating of features in the two dimensions of the image perpendicular to the line of sight of the camera, commonly the X and Y dimensions. In some embodiments, the image sensor may use range data as an input to focusing of the imaging sensor or to extraction of image data from selected focal planes. The range sensor provides range data 840. In some embodiments, the range sensor may use image data as an input to targeting of the range sensor or to extraction of range data.

In the embodiment of FIGS. 8 and 9, the flowchart of FIG. 9 shows a method suitable to the system of block diagram of FIG. 8. In the step “collect images” 910, collecting images can be implemented by the image sensor 810, possibly taking as input range data 840 passed via the computer vision analyzer 850 in an iterative and mode dependent process, and delivering as output image data 820 to the computer vision analyzer. The range sensor 830 also can possibly take image data 820 as input in order to specify range sensing requirements in an iterative and mode dependent process and in order to output range data 840. In the step “segment images” 920, segmenting images can be implemented by the image sensor 810 and by the computer vision analyzer 850, taking as input raw or processed image data from the image sensor and possibly also range data 840, for example in order to select focal planes. Segmenting images yields image data relevant to a subject and to body parts of a subject as input to calculating positions. In the step “calculate positions” 930, calculating positions can be implemented by the computer vision analyzer 850, taking as input image data and range data and possibly also with input from the posture analyzer 880 regarding required position data.

In an embodiment, “calculate positions” receives pixel data which can be acquired from a laterally positioned camera such as camera 235 in FIG. 2. The received pixel data can be segmented to remove pixels corresponding to stationary background elements of a scene. Thus the filtered pixel data can be assumed to correspond to a subject. A “calculate positions” computer algorithm can further process the pixel data by identifying the highest point of a subject, corresponding to the top of the subject's head. The algorithm can locate and follow the outline of a subject from the top toward the bottom, first following a path along the front of the subject and then following a path along the back of the subject. In following each path, the algorithm can identify and locate features such as the front of the face, the nose, the shoulders, the chest, the front of the torso, the pelvis, and the back. To facilitate identifying and locating features, the algorithm can define assumptions about the range of possible distances of a given feature from a reference point (for example the top of a subject's head), measuring the distance in a relative unit (for example a unit equaling a subject's front-to-back head width). For example, the shoulders can be assumed to be located within 1-2 head widths of the top of a subject's head. The algorithm can assign the best candidate location within a given range to the location or position of a given feature. Thus, the algorithm can calculate the positions of body parts.

In the step “calculate scalar posture data” 940, calculating scalar posture data can be implemented by the computer vision analyzer 850, possibly with input from the posture analyzer 880 regarding required scalar posture data 860. Calculate scalar posture data takes as input image data and range data reduced to position data and delivers scalar posture data 860 to the posture analyzer 880. In the step “analyze posture” 950, analyzing posture can be implemented by the posture analyzer 880, taking as input scalar posture data 860 and scalar thresholds 870. These data are analyzed and the results are delivered as input to a posture report 890. In step “report” 960, reporting also can be implemented by the posture analyzer 880 to deliver as output the posture report 890. At least some of the conclusions of an analysis of scalar posture data can be included in a posture report.

Looping, for example in FIGS. 5, 7, and 9, includes updating at least a portion of any output being reported. Updating can occur at a range of high frequencies from 60 Hz to 1 Hz or at lower frequencies in the range of once every second to once every day. Preferred frequencies include 60 Hz and 30 Hz. For example, updating may occur at a video rate of 30 times per second (Hz), resulting in continuous updating of the posture report as perceived in real time by a subject or other user. In an embodiment, when feedback is consistent and immediate, it can create a faster training cycle.

In another embodiment, subjects may respond to longer term feedback. For instance, reporting to a subject may include how much time the subject has maintained acceptable posture during an hour, a day, a week, or a month, thus providing positive feedback as average posture more closely aligns with ideal over time.

Different portions of a posture report may be updated to supply different stimuli or to supply different data storage requirements at different intervals. These options can be selectable by the subject or by another user.

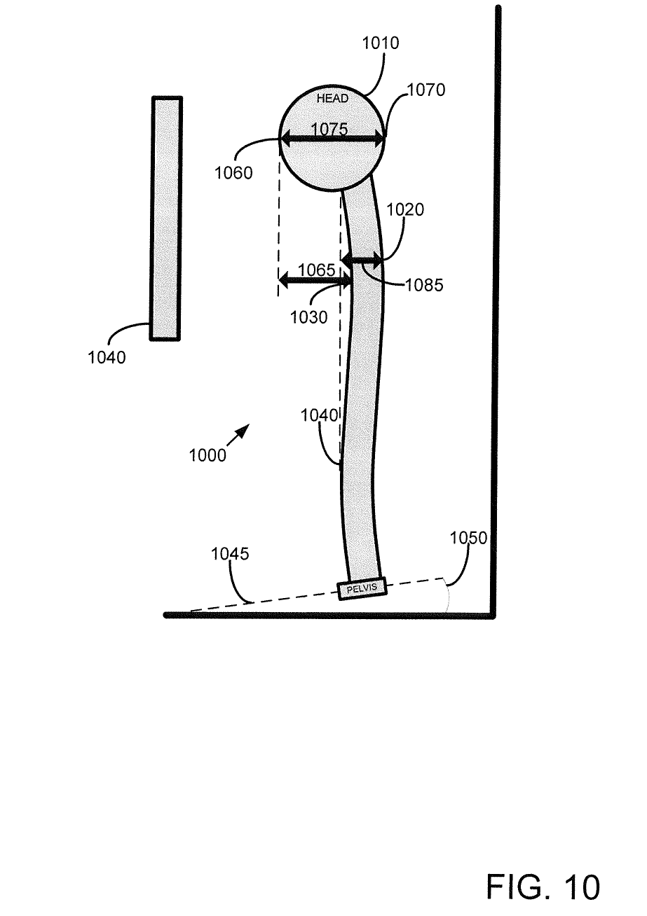

Referring now to FIG. 10, the diagram shows a schematic depiction 1000 of a subject positioned in front of a work surface 1040. FIG. 10 depicts how several example posture scalars can be calculated. The schematic 1000 of the subject additionally shows the head 1010 of the subject, the front of the head 1060, the back of the head 1070, the upper back 1020, the chest 1030, and the front of the pelvis 1040. In an embodiment, these body features can be identified within imaging data through image segmentation, for example in step 920 of FIG. 9 as implemented by a computer vision analyzer 850 in FIG. 8. After segmentation, positions of the body features can be calculated from range data, for example in step 930 of FIG. 9 as implemented by a range sensor 830 and/or a computer vision analyzer 850 in FIG. 8.

One example of a posture scalar is pelvic tilt, which can be calculated as the angle 1050 between horizontal and a line 1045 extending through the pelvis.

Another example of a posture scalar is head position, which can be calculated as the horizontal distance 1065 between the front of the head 1060 and the chest 1030, which can be normalized. Head position can be normalized by dividing the horizontal distance 1065 by a distance 1075 between the front of the head 1060 and the back of the head 1070, i.e. head position=(distance 1065)/(distance 1075).

Another example of a posture scalar is back curvature. In a preferred embodiment, back curvature can be measured as the horizontal component of the front to back distance between the rear most portion of a spine at a first height and the front most portion of the spine at a second, lower height, assuming a typical S-shape curvature to a spine. A greater horizontal distance corresponds to greater overall curvature of the spine. In an embodiment, the calculation can be corrected for an inclining or rotated overall body position to better measure curvature of the spine. In another embodiment, back curvature of a subject can be approximately calculated as the horizontal distance 1085 between the front of the torso 1040 and the back 1020 normalized by dividing by the distance 1075, i.e. back curvature=(distance 1085)/(distance 1075).

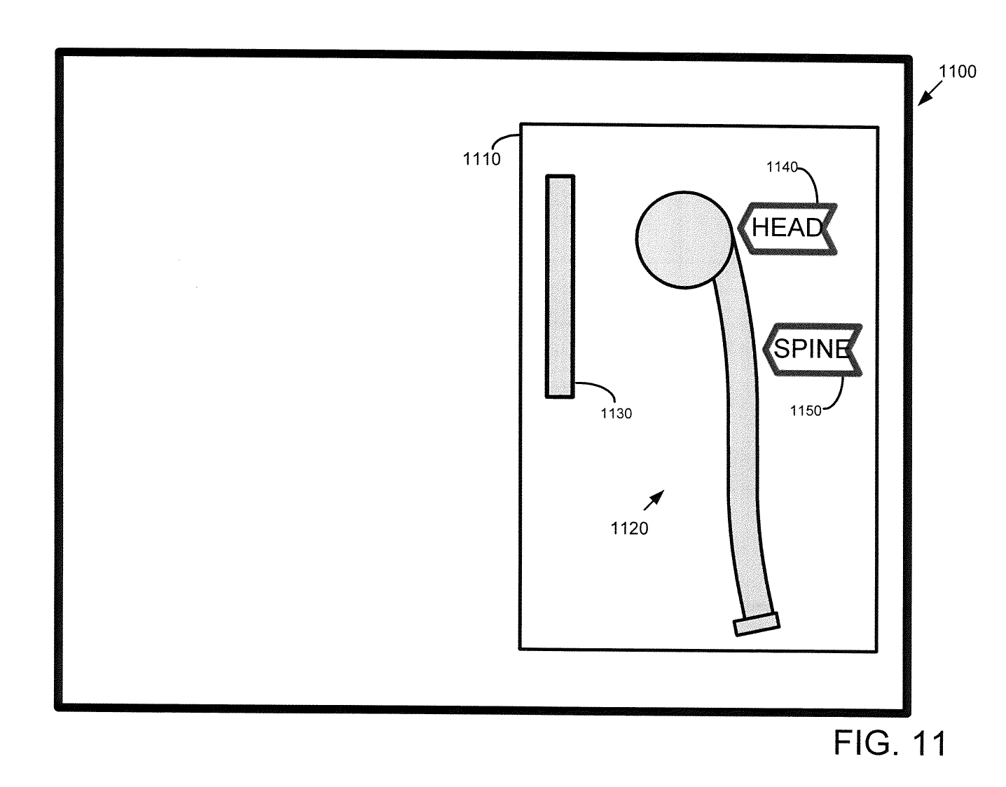

Referring now to FIGS. 8-11, in an embodiment, the diagram of FIG. 11 shows a schematic depiction 1100 of a computer display screen. A computer display screen is a type of electronic display. Additional examples of electronic displays useful in other embodiments include but are not limited to video monitors; television monitors; special purpose, limited-size, limited-resolution, limited-bit depth displays. Displayed within the screen 1100 is a computer graphics window 1110 generated by a graphical user interface. The window 1110 shows a schematic depiction 1120 of a subject positioned in front of a work surface 1130. The window is further populated with data from a posture report, for example posture report 890 generated in step 960. If, as a result of calculating head position, the posture analyzer 880 determines that a deficiency exists in the positioning of the head, then to the graphics window 1110 is added a head position deficiency marker 1140 as an example of a stimulus. Similarly, if, as a result of calculating back curvature, the posture analyzer 880 determines that a deficiency exists in the curvature of the back, then to the graphics window 1110 is added a spine curvature deficiency marker 1150 as another example of a stimulus. If the subject sees the markers, reacts to the feedback, corrects body positioning and posture, then, on a subsequent loop through the flowchart of FIG. 9, the posture analyzer can determine that a deficiency no longer exists and can, as appropriate, remove markers from being displayed. In some embodiments, the schematic 1120 of the subject can be adjusted over time to show the position of the subject's body parts, relative to each other and/or relative to the environment.

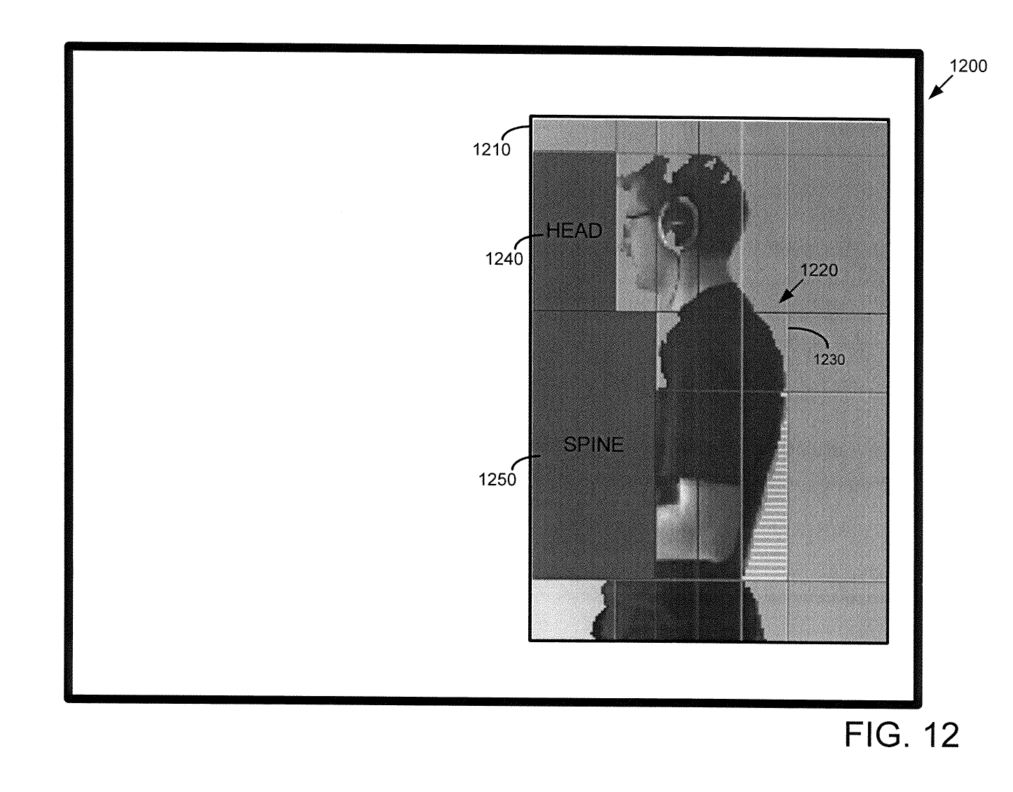

Similar to FIG. 11, the diagram of FIG. 12 shows a schematic depiction 1200 of a computer display screen. Displayed within the screen is a computer graphics window 1210 generated by a graphical user interface. The window 1210 shows an image 1220 of a subject. The subject may or may not be positioned in front of a work surface, and the work surface may or may not be shown. The window is further populated with data from a posture report, for example posture report 890 generated in step 960. If, as a result of calculating head position, the posture analyzer 880 determines that a deficiency exists in the positioning of the head, then to the graphics window 1210 is added a head position deficiency marker 1240, which can be a rectangle, possibly with a label “HEAD,” as an example of a stimulus. Similarly, if, as a result of calculating back curvature, the posture analyzer 880 determines that a deficiency exists in positioning of the curvature of the back, then to the graphics window 1210 is added a spine curvature deficiency marker 1250, which can be a rectangle, possibly with a label “SPINE,” as another example of a stimulus.

If the subject sees the markers, reacts to the feedback, corrects body positioning and posture, then, on a subsequent loop through the flowchart of FIG. 9, the posture analyzer can determine that a deficiency no longer exists and can as appropriate remove markers from being displayed. In some embodiments, the image 1220 of the subject can be a live image which updates over time to show the position of the subject's body parts, relative to each other and/or relative to the environment. In another embodiment, the display window 1210 can also show segmentation indices used by the computer vision analyzer 850 to extract features such as body parts from the image data. A segmentation index 1230 indicating feature extraction and the position of the back appears in window 1210.

### Exemplary Computing Environment

The techniques and solutions described herein can be performed by software, hardware, or both as elements of a computing environment, such as one or more computing devices. For example, computing devices include server computers, desktop computers, laptop computers, notebook computers, handheld devices, netbooks, tablet devices, mobile devices, PDAs, and other types of computing devices.

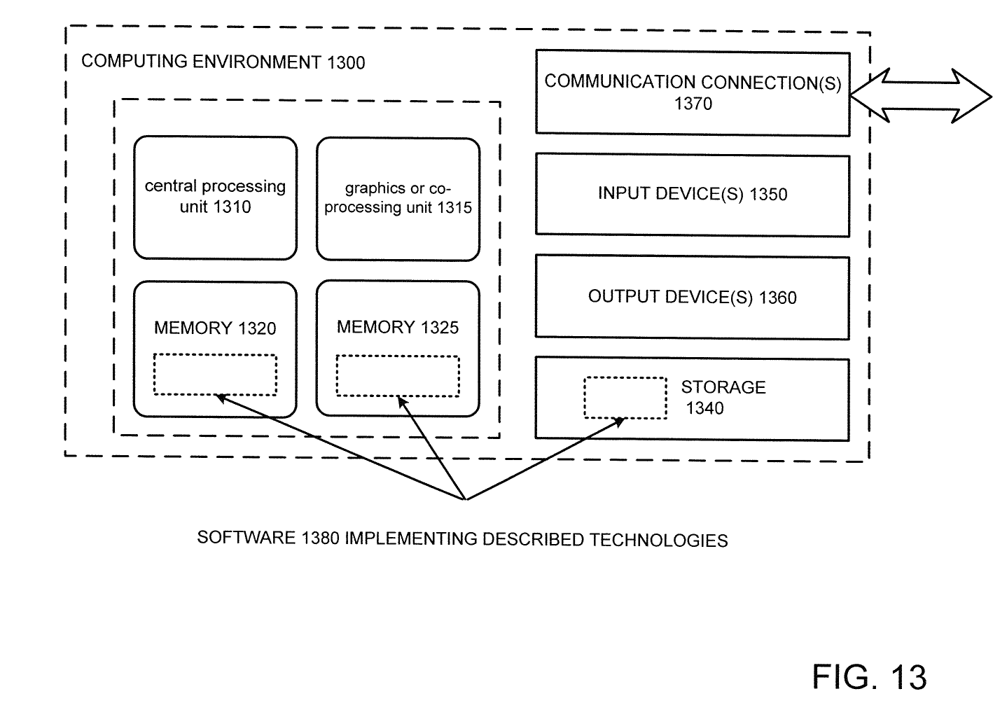

FIG. 13 illustrates a generalized example of a suitable computing environment 1300 in which the described technologies can be implemented. The computing environment 1300 is not intended to suggest any limitation as to scope of use or functionality, as the technologies may be implemented in diverse general-purpose or special-purpose computing environments. For example, the disclosed technology may be implemented using a computing device comprising a processing unit, memory, and storage storing computer-executable instructions implementing the enterprise computing platform technologies described herein. The disclosed technology may also be implemented with other computer system configurations, including hand held devices, multiprocessor systems, microprocessor-based or programmable consumer electronics, network PCs, minicomputers, mainframe computers, a collection of client/server systems, and the like. The disclosed technology may also be practiced in distributed computing environments where tasks are performed by remote processing devices that are linked through a communications network. In a distributed computing environment, programs and program modules may be located in both local and remote memory storage devices.

With reference to FIG. 13, the computing environment 1300 includes at least one processing unit 1310 coupled to memory 1320. In FIG. 13, this basic configuration 1330 is included within a dashed line. The processing unit 1310 executes computer-executable instructions and may be a real or a virtual processor (e.g., executing on one or more hardware processors). In a multi-processing system, multiple processing units execute computer-executable instructions to increase processing power. The memory 1320 may be volatile memory (e.g., registers, cache, RAM), non-volatile memory (e.g., ROM, EEPROM, flash memory, etc.), or some combination of the two. The memory 1320 can store software 1380 implementing any of the technologies described herein.

A computing environment may have additional features. For example, the computing environment 1300 includes storage 1340, one or more input devices 1350, one or more output devices 1360, and one or more communication connections 1370. An interconnection mechanism (not shown) such as a bus, controller, or network interconnects the components of the computing environment 1300. Typically, operating system software (not shown) provides an operating environment for other software executing in the computing environment 1300, and coordinates activities of the components of the computing environment 1300.

The storage 1340 may be removable or non-removable, and includes magnetic disks, magnetic tapes or cassettes, CD-ROMs, CD-RWs, DVDs, or any other computer-readable media which can be used to store information and which can be accessed within the computing environment 1300. The storage 1340 can store software 1380 containing instructions for any of the technologies described herein.

The input device(s) 1350 may be a touch input device such as a keyboard, mouse, pen, or trackball, a voice input device, a scanning device, or another device that provides input to the computing environment 1300. For audio, the input device(s) 1350 may be a sound card or similar device that accepts audio input in analog or digital form, or a CD-ROM reader that provides audio samples to the computing environment. The output device(s) 1360 may be a display, printer, speaker, CD-writer, or another device that provides output from the computing environment 1300.

The communication connection(s) 1370 enable communication over a communication mechanism to another computing entity. The communication mechanism conveys information such as computer-executable instructions, audio/video or other information, or other data. By way of example, and not limitation, communication mechanisms include wired or wireless techniques implemented with an electrical, optical, RF, infrared, acoustic, or other carrier.

The techniques herein can be described in the general context of computer-executable instructions, such as those included in program modules, being executed in a computing environment on a target real or virtual processor. Generally, program modules include routines, programs, libraries, objects, classes, components, data structures, etc., that perform particular tasks or implement particular abstract data types. The functionality of the program modules may be combined or split between program modules as desired in various embodiments. Computer-executable instructions for program modules may be executed within a local or distributed computing environment.

Any of the computer-readable media herein can be non-transitory (e.g., memory, magnetic storage, optical storage, or the like).

Any of the storing actions described herein can be implemented by storing in one or more computer-readable media (e.g., computer-readable storage media or other tangible media).

Any of the things described as stored can be stored in one or more computer-readable media (e.g., computer-readable storage media or other tangible media).

Any of the methods described herein can be implemented by computer-executable instructions in (e.g., encoded on) one or more computer-readable media (e.g., computer-readable storage media or other tangible media). Such instructions can cause a computer to perform the method. The technologies described herein can be implemented in a variety of programming languages.

Any of the methods described herein can be implemented by computer-executable instructions stored in one or more computer-readable storage devices (e.g., memory, magnetic storage, optical storage, or the like). Such instructions can cause a computer to perform the method.

### ALTERNATIVES

The technologies from any example can be combined with the technologies described in any one or more of the other examples. In view of the many possible embodiments to which the principles of the disclosed technology may be applied, it should be recognized that the illustrated embodiments are examples of the disclosed technology and should not be taken as a limitation on the scope of the disclosed technology. Rather, the scope of the disclosed technology includes what is covered by the following claims. I therefore claim all that comes within the scope and spirit of the claims.

I claim:

1. A method for improving posture, the method comprising the steps of:
acquiring data about a subject using sensing equipment spaced apart from the subject;
making a comparison to threshold posture data; and
reporting results of the comparison.

2. The method of claim 1, wherein the sensing equipment is at least 2 feet away from the subject.

3. The method of claim 1, wherein reporting results includes displaying computer graphics on an electronic display.

4. The method of claim 3, wherein reporting results includes displaying at least a portion of an image of the subject.

5. The method of claim 1, further comprising repeating the steps of acquiring data, making a comparison, and reporting the results at a frequency of at least once per second.

6. The method of claim 1, wherein results are reported to a user other than the subject.

7. The method of claim 1, wherein a posture program executes one or more steps of the method, and wherein that program runs in a multi-threaded computational environment and runs in background while another program runs in foreground.

8. The method of claim 1, wherein the data acquired is image data from which is extracted scalar posture data.

9. The method of claim 1, wherein making a comparison comprises determining whether at least two body parts are positioned correctly, and wherein reporting results includes reporting a posture deficiency if the at least two body parts are not positioned correctly.

10. The method of claim 1, wherein at least a part of the hardware or software of a posture estimation system performs at least a part of the acquiring data or making a comparison steps.

11. A system for improving posture, the system comprising:
sensing equipment, wherein the sensing equipment acquires data about a subject and wherein the sensing equipment is positioned remotely from the subject;
an analyzer, wherein the analyzer makes a comparison to threshold posture data and outputs one or more results of the comparison as a report.

12. The system of claim 1, wherein the sensing equipment is at least two feet away from the subject.

13. The system of claim 1, wherein the output report includes computer graphics on an electronic display.

14. The system of claim 13, wherein the output report includes at least a portion of an image of the subject.

15. The system of claim 1, wherein at least a portion of the output report is updated at a frequency of at least once per second.

16. The system of claim 1, wherein at least a portion of the output report is received by a user other than the subject.

17. The system of claim 1, wherein the analyzer is a program or part of a program, and wherein that program runs in a multi-threaded computational environment and runs in background while another program runs in foreground.

18. The system of claim 1, wherein the data acquired is image data from which is extracted scalar posture data.

19. The system of claim 1, wherein two or more body locations all must be positioned correctly to avoid a posture deficiency in the output report.

20. The system of claim 1, wherein at least a part of the hardware or software of a posture estimation system provides at least a part of the sensing equipment or the analyzer.
